(19) United States
(12) Patent Application Publication
(10) Pub. No.: US 2010/0185052 A1
(43) Pub. Date: Jul. 22, 2010

(54) Endoscopic device

(76) Inventor: Hui-Yu Chang, Taipei (TW)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/657,342

(22) Filed: Jan. 19, 2010

(30) Foreign Application Priority Data
Jan. 19, 2009 (TW) 098101913

(51) Int. Cl. A61B 1/04

(52) U.S. Cl. 600/112

(57) ABSTRACT

An endoscopic device includes a flexible PCB adapted to fold to form a stacked structure with a plurality of spaced layers. The PCB comprises a plurality of units, at least one interconnection interconnecting any two adjacent units, and a plurality of connections connecting to a conductor. Advantageously, the connections can reduce space for mounting electronic devices on the units. Each of the connections has a first section extending from the connected unit, and a second section extending from the first section with a passive electronic component being disposed thereon. Further, a conductor extends from the second section of the connection.

### PRIORITY AND CROSS-REFERENCE TO RELATED APPLICATION

This application claims foreign priority benefit under 35 U.S.C. §119 of Taiwanese Patent Application No. 098101913 and filed Jan. 19, 2009.

### BACKGROUND OF THE INVENTION

1. Field of Invention

The invention relates to endoscopic devices.

2. Description of Related Art

Endoscopic tools for conducting a medical examination within the human body cavities are well known in the art. For example, U.S. Publication Application No. 2006/0189844 discloses an endoscopic device. Drawbacks thereof are described below. Areas for soldering on a PCB (printed circuit board) are required. This makes a reduction of PCB size impossible. Further, a length about 30 mm is formed by folding buses. Such length can adversely limit a moving range of the endoscopic device within the body organs. For example, a doctor cannot observe larynx and trachea by means of the endoscopic device.

The above drawbacks are common among typical endoscopic devices and are not solved. Thus, continuing improvements in the exploitation of endoscopic device are constantly being sought.

### SUMMARY OF THE INVENTION

It is therefore one object of the invention to provide an endoscopic device having a cover with a diameter less than 5 mm, for example. The endoscopic device is cost effective and can be discarded after use so as to prevent cross-infection from occurring.

Preferably, the endoscopic device has a diameter less than 4.3 mm.

It is another object of the invention to provide an endoscopic device having a lens with a length less than 10 mm. Lens Length is an important factor for determining quality of the endoscopic device. In general, a shorter lens makes the endoscopic device easier to be inserted into narrow body cavities for medical examination. The lens length is preferably about 10 mm.

Preferably, the endoscopic device comprises a flexible PCB adapted to fold to form a stacked structure with a plurality of spaced layers. The PCB comprises a plurality of units, at least one interconnection interconnecting any two adjacent units, and a plurality of connections connecting to a conductor. Advantageously, the connections can reduce space for mounting electronic devices on the units. Each of the connections comprises a first section extending from the connected unit, and a second section extending from the first section with a passive electronic component being disposed thereon. Further, a conductor extends from the second section of the connection.

Preferably, the interconnections have a length about the same as the height of two adjacent units being bent. This enables the PCB to fold as a stacked structure with a plurality of spaced layers. The stacked structure is an unbending section. The connections are adapted to extend downward from the stacked structure. The first section of the connection has a length greater than 3 mm of the height of the unbending section.

Preferably, larger active electronic components such as microcontrollers and sensors are mounted in the units, whereas smaller passive electronic components such as resistors and capacitors are mounted in the second sections of the connections. This eliminates drawbacks of the prior art endoscopic device which comprise active and passive electronic components being mounted in the PCB, making it difficult to reduce the size of the PCB and the endoscope device itself.

Preferably, the first sections of the connections have a length greater than the height of the unbending section so that electronic components mounted on the first sections of the connections can be disposed externally of the stacked units. A conductor is extended from the second section of each connection for signal transmission. Each of the interconnections has a width about 2 mm, for example. Each of the connections has a width about 2 mm, for example. Each of the interconnections and the connections extends from the periphery of the unit. Thus, after folding, the interconnections and the connections do not substantially increase the size of the lens. This is one of the advantages of the invention.

With comparison to the prior art, the invention has the following advantages:

The units of the flexible PCB have larger active electronic components such as microcontrollers and sensors mounted thereon, whereas the smaller passive electronic components are mounted on the connections. Hence, it can reduce the PCB size to a minimum.

The active electronic components mounted on the units can be in signal communication with the external devices via the conductors extending out of the second sections of the connection. Hence, a reduction of conductor length is made possible. Further, layout area can be distributed, resulting in a size reduction of the PCB including the units.

The bending angle of each of the interconnections and the connections is a minimum so as to greatly decrease the PCB size and the diameter of the endoscope device.

The units with active electronic components mounted thereon can be folded to form a stacked structure because the interconnections have a sufficient length to be bent. This has the effects of greatly decreasing the height of the unbending section and increasing the operating space of the endoscopic device so that the device can be used for examining larynx and trachea, for example.

The connections and conductors (e.g., small wires) extending therefrom are flexible in nature. This means that except the unbending section they are bendable. Further, their bending angles are much smaller than that of the conventional fiber optic camera.

The above and other objects, features and advantages of the invention will become apparent from the following detailed description taken with the accompanying drawings.

### BRIEF DESCRIPTION OF THE DRAWINGS

FIG. 1 is an exploded view of a first preferred embodiment of endoscopic device according to the invention;

FIG. 2 is a perspective view of the assembled endoscopic device of FIG. 1;

FIG. 3 is a top plan view of the fully extended PCB of FIG. 1;

FIG. 3A is a side elevation of the first and the second unit of FIG. 3 upon being folded;

FIG. 4 is a top plan view of the fully extended PCB of a second preferred embodiment of endoscopic device;

FIG. 4A is a side elevation of the first and the second unit of FIG. 4 upon being folded;

FIG. 5 is another top plan view of the fully extended PCB of the second preferred embodiment;

FIG. 5A is a side elevation of the first and the second unit of FIG. 5 upon being folded;

FIG. 6 is a perspective view of the PCB of the second preferred embodiment;

FIG. 7 is a top plan view of the fully extended PCB of a third preferred embodiment of endoscopic device;

FIG. 7A is a side elevation of the first and the second unit of FIG. 7 upon being folded;

FIG. 8 is another top plan view of the fully extended PCB of the third preferred embodiment;

FIG. 8A is a side elevation of the first and the second unit of FIG. 8 upon being folded;

FIG. 9 is an exploded view of a third preferred embodiment of endoscopic device; and

FIG. 10 is a perspective view of the assembled endoscopic device of FIG. 9.

### DETAILED DESCRIPTION OF THE INVENTION

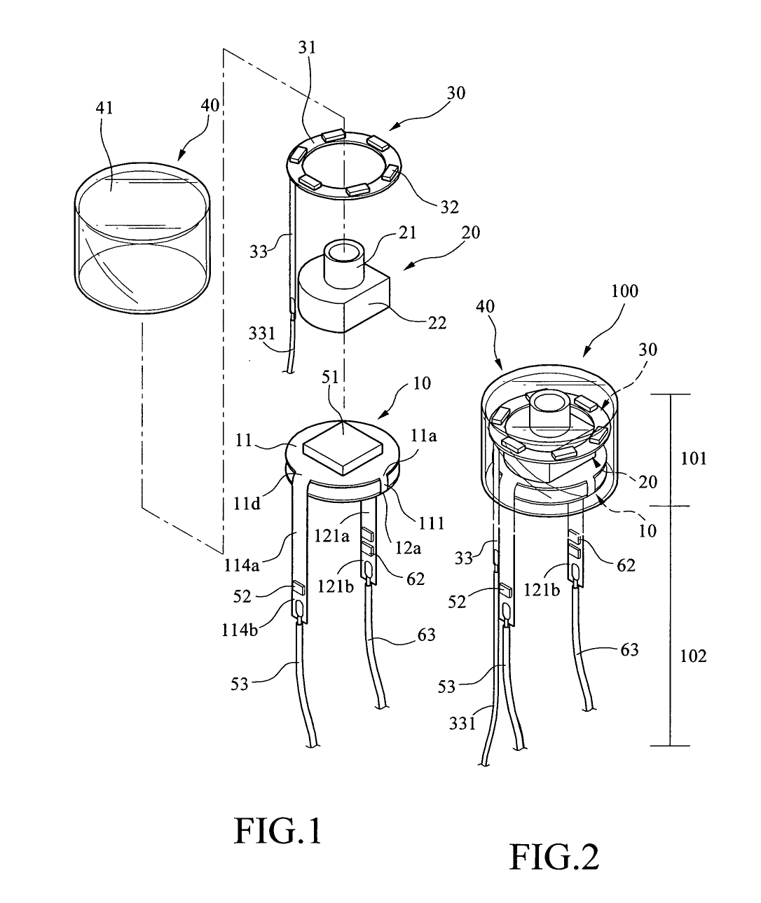

Referring to FIGS. 1, 2, 3 and 3A, an endoscopic device 100 in accordance with a first preferred embodiment of the invention comprises the following components as discussed in detail below.

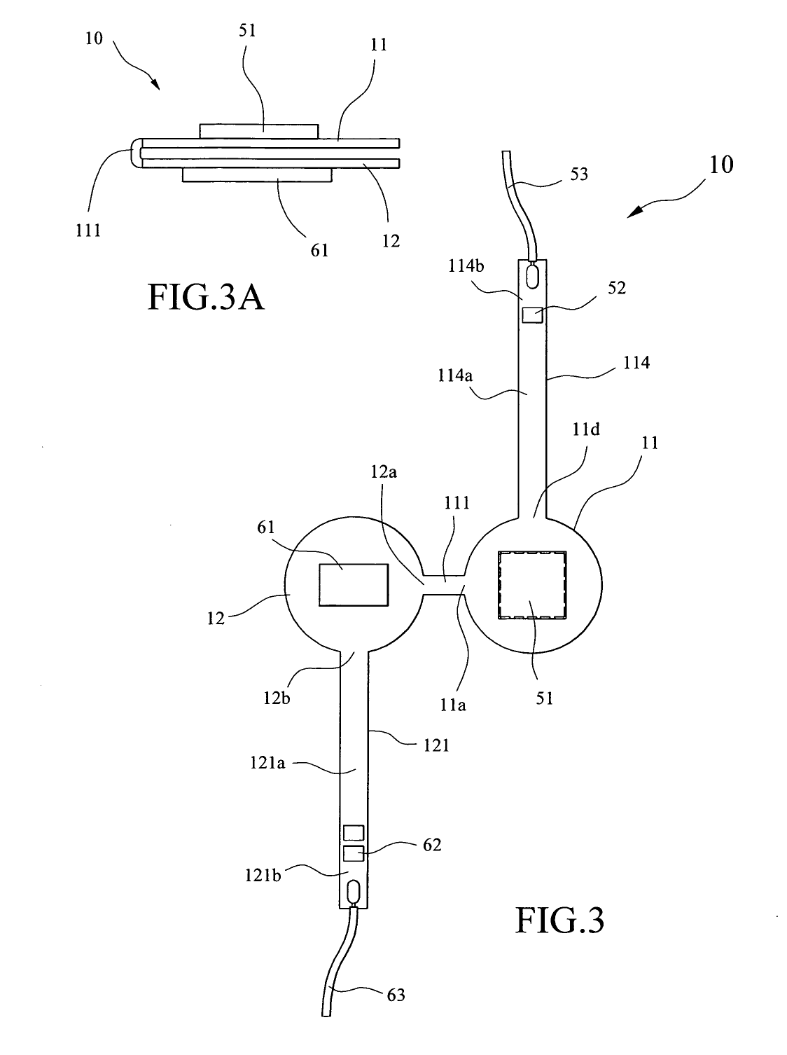

A flexible PCB 10 can be folded to form a layered stack with two spaced layers (see FIG. 3A). An image capturing assembly 20 for image capture and processing comprises a wedge-shaped seat 22 and a cylindrical lens 21 provided on the seat 22. A light source 30 which is disposed on or above the seat 22 comprises a ring-shaped electronic member 31, a plurality of light emitting elements (e.g., LEDs (light-emitting diodes) or OLED (organic light-emitting diodes)) 32 provided on the electronic member 31, and a wire 33 extending downward out of the bottom of the electronic member 31. An electrical conductor 331 is connected to one end of the wire 33 for providing electric power to the light emitting elements 32. The seat 22 is seated on the PCB 10. The electronic member 31 is rested upon the seat 22 with the lens 21 passing through a hole in the seat 22.

An inverted cup-shaped cover 40 is put on the assembled PCB 10, the image capturing assembly 20, and the light source 30. The cover 40 has a transparent member 41 on a top so that light emitted by the light emitting elements 32 can pass through the transparent member 41 and external light passing the transparent member 41 can be captured by the lens 21. The cover 40 can have a diameter less than 5 mm, for example. More preferably, the cover 40 has a diameter less than 4.3 mm.

As shown in FIG. 2, the assembled endoscopic device 100 is composed of an unbending section 101 and a bendable section 102. Portions of the PCB 10, the image capturing assembly 20, and the light source 30 in the cover 40 are disposed in the unbending section 101. Preferably, the unbending section 101 has a length less than 10 mm, for example.

As shown in FIG. 3, the PCB 10 comprises a first unit 11, and a second unit 12 interconnected together. Each unit can be shaped as a disc, for example.

The first unit 11 comprises a bridge point 11a, and a connecting point 11d spaced around a periphery thereof, the second unit 12 comprises a bridge point 12a and a connecting point 12b spaced from the bridge point 12a about 90 degrees on a periphery thereof. A first interconnection 111 is interconnected between the bridge points 11a and 12a, and has a width of about 2 mm or less, for example.

As shown in FIGS. 1 and 3A, a first active electronic component 51 is provided on top the first unit 11 and a second active electronic component 61 is provided on underside of the second unit 12. The first active electronic component 51 is provided within the seat 22. The second unit 12 is provided under the first unit 11 by bending the first interconnection 111. The first active electronic component 51 may comprise an imaging sensor and the second active electronic component 61 may comprise a microcontroller.

Alternatively, the second active electronic component 61 is provided on top of the second unit 12 (figure not shown).

In FIG. 3, first connection 114 is extended from the connecting point 11d, a second connection 121 is extended from the connecting point 12b respectively.

The first connection 114 comprises a first section 114a extending from the connecting point 11d, and a second section 114b extending from the first section 114a. A first passive electronic component 52 is provided on the second section 114b.

The second connection 121 comprises a first section 121a extending from the connecting point 12b, and a second section 121b extending from the first section 121a. Two second passive electronic components 62 are provided on the second section 121b. The first and second passive electronic components 52, 62 may comprise resistors, capacitors or the like. In the first embodiments, the width of the first and second connections 114, 121 allow reducing the size of the PCB10 since each connection is adapted to smoothly bend from the periphery of the unit without unnecessary arcuate portion (as shown in FIG. 1). The second sections 114b, 121b can further reduce the size of the endoscopic device 100 since the passive electronic components 52, 62 are removed from the first and second units 11, 12.

The first and second connections 114, 121 together with the wire 33 of the light source 30 form the bendable section 102 of the endoscopic device 100 (see FIG. 2).

A conductor 53 is extended from the second section 114b ; a conductor 63 is extended from the second section 121 b respectively. Preferably, the first and second sections 121a, 114a both have a length greater than a distance between the first unit 11 and the second unit 12 to keep the passive electronic components 52, 62 out of the unbending section 101 as show in FIG. 2.

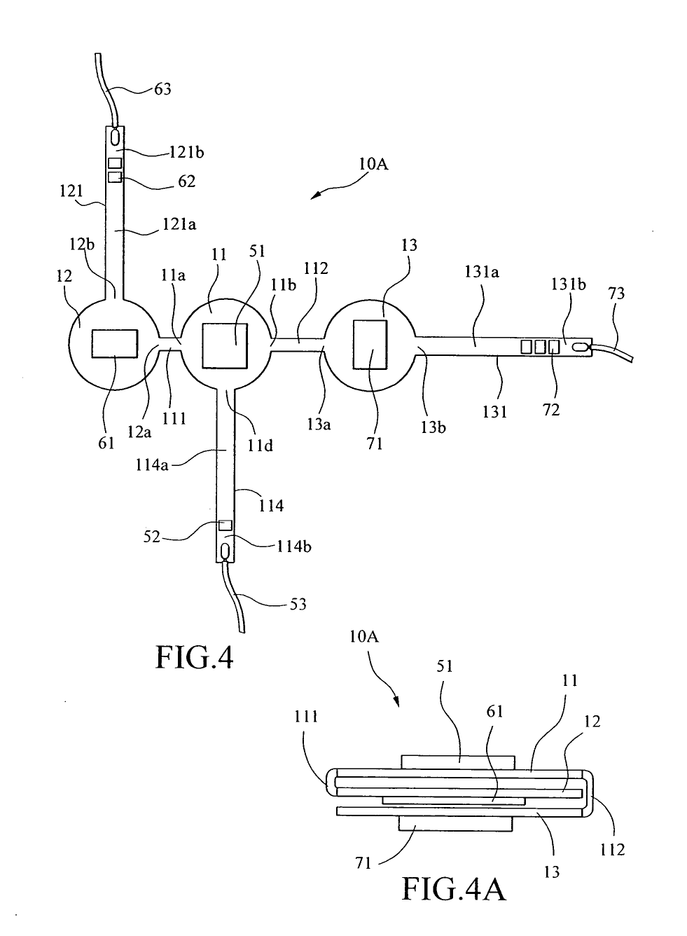

Referring to FIGS. 4, 4A, 5, 5A, and 6, a PCB of the endoscopic device 100 in accordance with a second preferred embodiment of the invention comprises the following components as discussed in detail below.

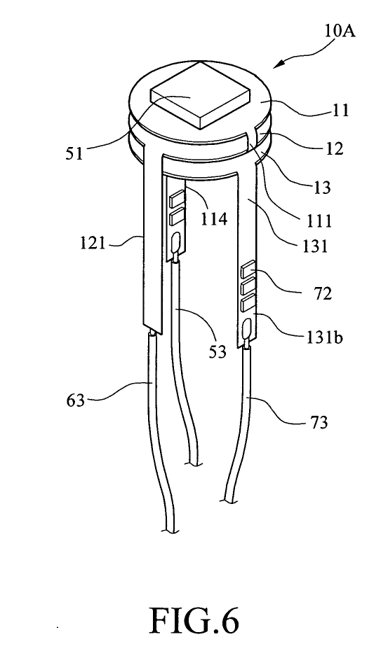

As shown in FIGS. 4 and 4A, the PCB 10A not only comprises a first unit 11, and a second unit 12 the same as the first preferred embodiment, but also further includes a third unit 13. The PCB 10A can be folded to form a three spaced layered stack as shown in FIG. 6. Note that each unit is shaped as a disc.

The third unit 13 comprises a bridge point 13a and a connecting point 13b opposing the bridge point 13a on a periphery thereof, a second interconnection 112 is interconnected between the bridge points 13a and a bridge point 11b in the first unit 11, and a third connection 131 is extended from the connecting point 13b.

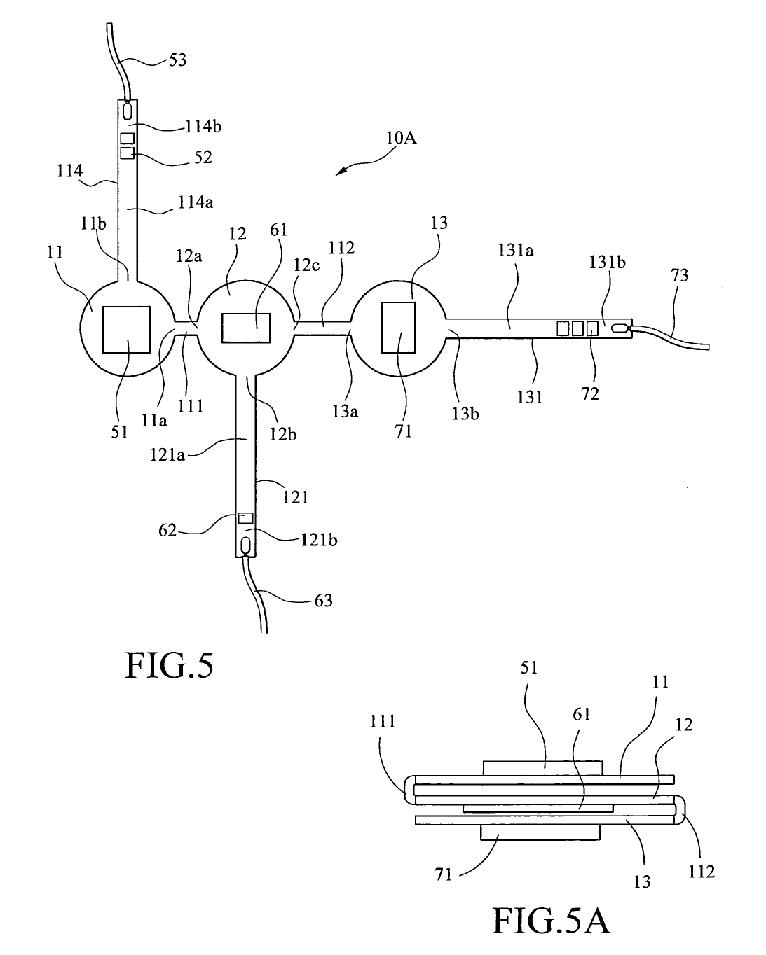

Alternatively, the second interconnection 112 can be interconnected the bridge point 13a of the third unit 13 and a bridge point 12c of the second unit 12 as shown in FIGS. 5. 5A.

As shown in FIGS. 4A and 5A, third active electronic component 71 is provided on the underside side of the third unit 13 which is provided under the second active electronic component 61 by bending the second interconnection 112. This is a stacked structure with three spaced layers. Alternatively, the third active electronic component 71 can be provided on top of the third unit 13.

As shown in FIGS. 4 and 5, the third connection 131 comprises a first section 131a extending from the connecting point 13b, and a second section 131b extending from the first section 131a. Preferably, the first section 131a has a length greater than a distance between the first unit 11 and the third unit 13 to keep the passive electronic components 72 out of the non-bending section 101.

A third passive electronic component 72 is provided on the second section 131b, and a conductor 73 is extended from the second section 131b.

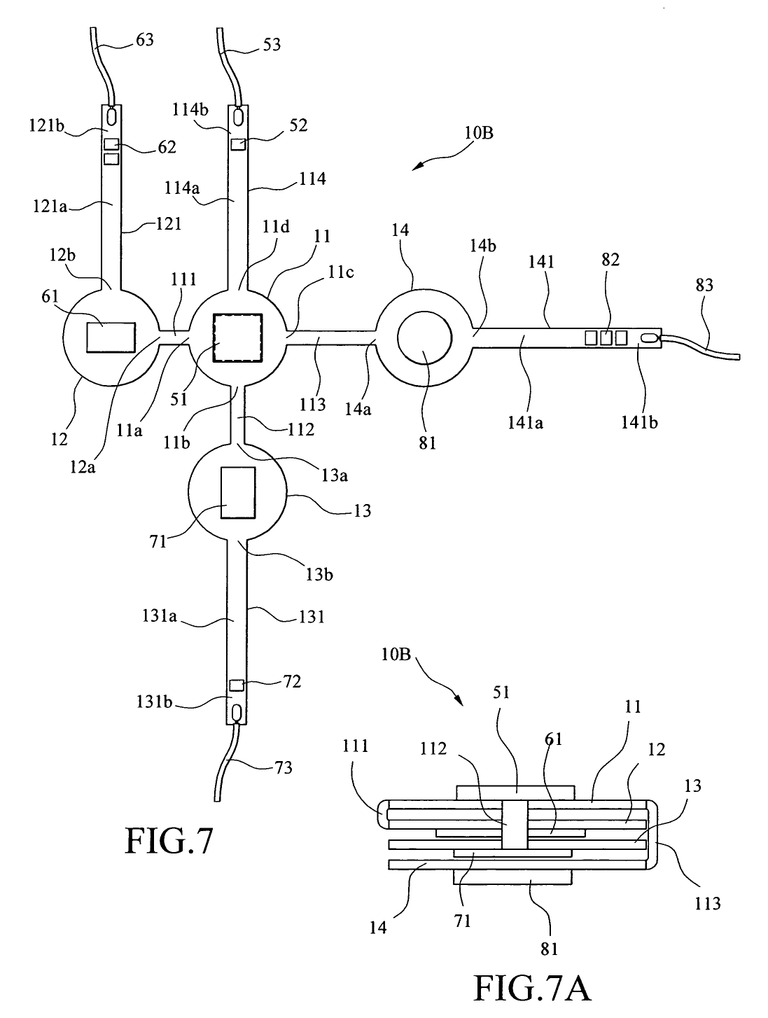

Referring to FIGS. 7, 7A, 8, 8A, 9 and 10, an endoscopic device 100 and PCB thereof in accordance with a third preferred embodiment of the invention comprises the following components as discussed in detail below.

As shown in FIGS. 7 and 7A, the PCB 10B not only comprises a first unit 11, a second unit 12 and a third unit 13 the same as the second preferred embodiment, but further includes a fourth unit 14. The PCB 10B can be folded to form a four spaced layered stack. Note that each unit is shaped as a disc, for example.

The fourth unit 14 comprises a bridge point 14a and a connecting point 14b opposing the bridge point 14a on a periphery thereof, a third interconnection 113 is interconnected between the bridge point 14a and a bridge point 11c in the first unit 11, a fourth connection 141 is extended from the connecting point 14b.

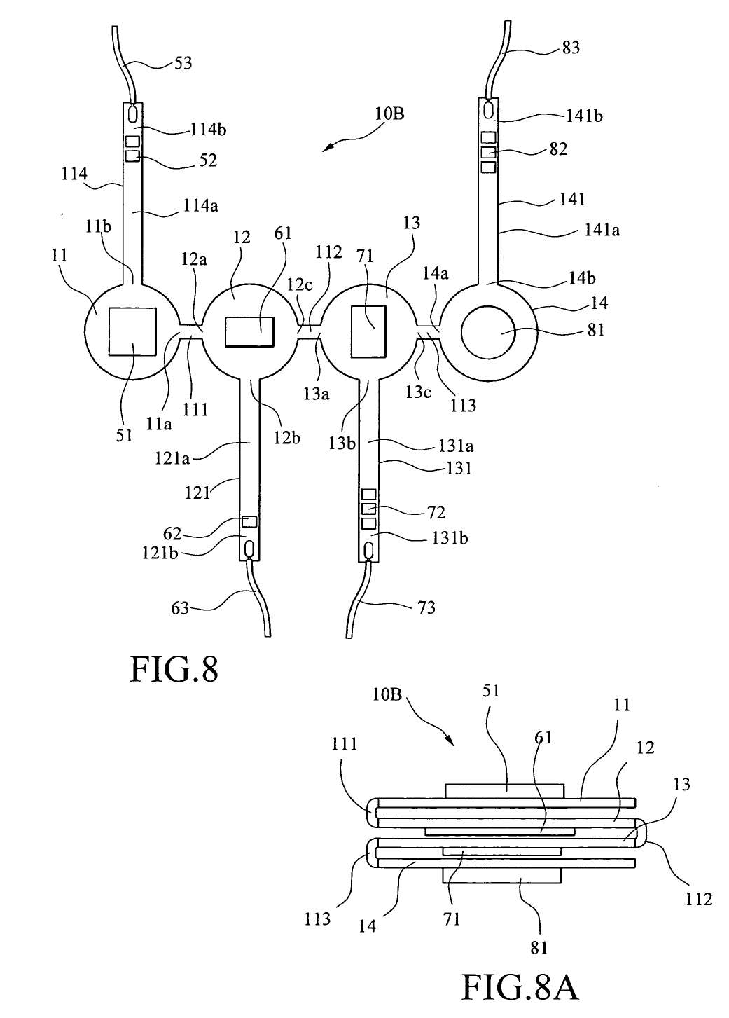

Alternatively, the third interconnection 113 can be interconnected the bridge point 14a of the fourth unit 14 and a bridge point 13c of the third unit 13 as shown in FIGS. 8. 8A.

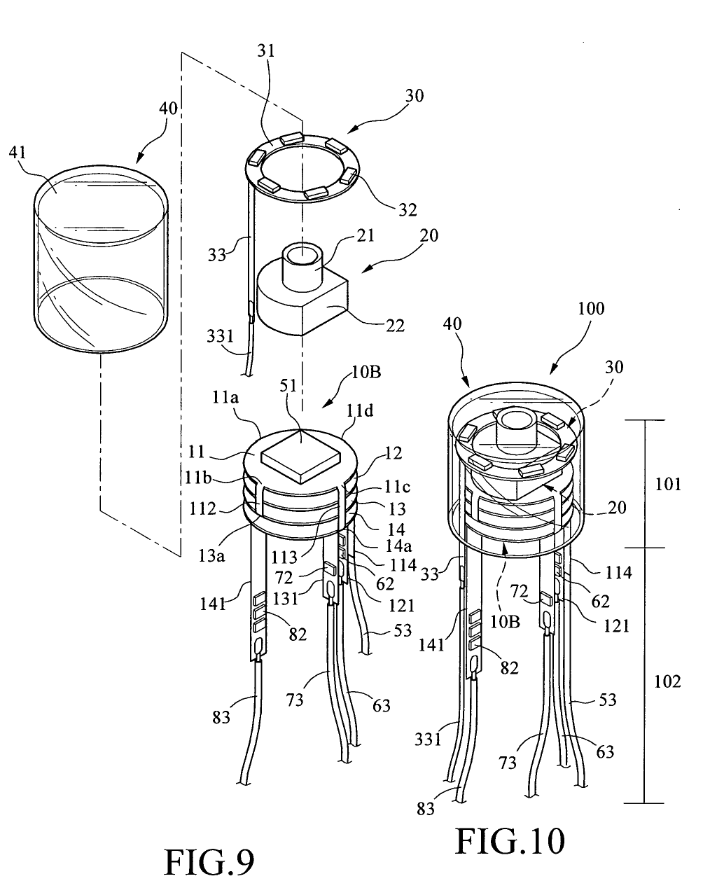

A fourth active electronic component 81 is provided on the underside of the fourth unit 14 which is provided under the third active electronic component 71 by bending the third interconnection 113. This is a stacked structure with four spaced layers as shown in FIG. 9. Alternatively, the fourth active electronic component 81 is provided on top of the fourth unit 14.

As shown in FIGS. 7 and 8, the fourth connection 141 comprises a first section 141a extending from the connecting point 14b, and a second section 141b extending from the first section 141a. Preferably, the first section 141a has a length greater than a distance between the first unit 11 and the fourth unit 14 to keep the passive electronic components 82 out of the non-bending section 101 as shown in FIG. 10.

A fourth passive electronic component 82 is provided on the second section 141b, and a conductor 83 is extended from the second section 141b.

In the above embodiments, each of the units has a diameter of 3.7 mm, for example, and each of the connections has a width of about 2 mm or less, for example. The width of the connections allows the reduction of the size of the PCB since each connection is adapted to smoothly bend from the periphery of the unit without unnecessary arcuate portion.

While two units, three units and four units are discussed above in the first, second and third embodiments respectively, however, if micro-electronic technology is further upgraded, a fourth preferred embodiment of the endoscopic device of the present invention having only one unit (not shown) is available to attain the same function and purpose of the present invention as mentioned above.

While the invention herein disclosed has been described by means of specific embodiments, numerous modifications and variations could be made thereto by those skilled in the art without departing from the scope of the invention set forth in the claims.

What is claimed is:

1. An endoscopic device (100) comprising:
an image capturing assembly (20) comprising a seat (22) and a lens (21) disposed on the seat (22);
a light source (30) disposed in relationship to the seat (22) for light emission;
a flexible circuit board (10) comprising:
a first unit (11) having a first side and an opposing second side, the first unit comprising a first active electronic component (51) and the image capturing assembly (20) disposed on the first side for image capture and processing, the first unit (11) further comprising a first bridge point (11a) and a connecting point (11d) spaced around a periphery thereof;
a second unit (12) comprising a first bridge point (12a) and a connecting point (12b), both being on a periphery thereof;
a first interconnection (111) interconnecting the first bridge point (11a) of the first unit (11) and the first bridge point (12a) of the second unit (12);
a first connection (114) comprising a first section (114a) extending from the connecting point (11d) of the first unit (11), and a second section (114b) extending from the first section (114a) with a first passive electronic component (52) disposed thereon;
a second connection (121) comprising a first section (121a) extending from the connecting point (12b) of the second unit (12), and a second section (121b) extending from the first section (121a) with a second passive electronic component (62) disposed thereon;
wherein the second unit (12) is disposed under the first unit (11), facing the second side of the first unit, by bending the first interconnection (111) so as to shape the circuit board (10) into a two layered structure;
a cover (40) including a transparent member (41), facing the first side of the first unit for covering the layered structure, and aligned with the image capturing assembly (20).

2. The endoscopic device (100) of claim 1, wherein the first sections (121a, 114a) both have a length greater than a distance between the first unit (11) and the second unit (12).

3. The endoscopic device (100) of claim 1, further comprising a second active electronic component (61) disposed on the second unit (12).

4. The endoscopic device (100) of claim 1, wherein the first interconnections (111), the first connection (114), and the second connection (121) all have a width about 2 mm, and the cover (40) has a diameter less than 5 mm.

5. An endoscopic device (100) comprising:
an image capturing assembly (20) comprising a seat (22) and a lens (21) disposed on the seat (22);
a light source (30) disposed in relationship to the seat (22) for light emission;
a flexible circuit board (10A) comprising:
a first unit (11) having a first side and an opposing second side, the first unit comprising a first active electronic component (51) and the image capturing assembly (20) disposed on the first side for image capture and processing, the first unit (11) further comprising first, and second bridge points (11a, 11b) and a connecting point (11d) spaced around a periphery thereof;
a second unit (12) comprising a first bridge point (12a) and a connecting point (12b), both being on a periphery thereof;
a third unit (13) comprising a first bridge point (13a) and a connecting point (13b), both being on a periphery thereof;
a first interconnection (111) interconnecting the first bridge point (11 a) of the first unit (11) and the first bridge point (12a) of the second unit (12);
a second interconnection (112) interconnecting the second bridge point (11b) of the first unit (11) and the first bridge point (13a) of the third unit (13);
a first connection (114) comprising a first section (114a) extending from the connecting point (11d) of the first unit (11), and a second section (114b) extending from the first section (114a) with a first passive electronic component (52) disposed thereon;
a second connection (121) comprising a first section (121a) extending from the connecting point (12b) of the second unit (12), and a second section (121b) extending from the first section (121a) with a second passive electronic component (62) disposed thereon;
a third connection (131) comprising a first section (131a) extending from the connecting point (13b) of the third unit (13), and a second section (131b) extending from the first section (131a) with a third passive electronic component (72) disposed thereon;
wherein the second unit (12) is disposed under the first unit (11), facing the second side of the first unit, by bending the first interconnection (111), the third unit (13) is disposed under the second unit (12) facing a second side of the second unit further away from the first unit by bending the second interconnection (112) so as to shape the circuit board (10A) into a three layered structure;
a cover (40) including a transparent member (41), facing the first side of the first unit for covering the three layered structure, and aligned with the image capturing assembly (20).

6. The endoscopic device (100) of claim 5, wherein the first section (121a, 114a) both have a length greater than a distance between the first unit (11) and the second unit (12); the first section (131a) has a length greater than a distance between the first unit (11) and the third unit (13).

7. The endoscopic device (100) of claim 5, further comprising a second active electronic component (61) disposed on the second unit (12), and a third active electronic component (71) disposed on the third unit (13).

8. The endoscopic device (100) of claim 5, wherein the cover (40) has a diameter less than 5 mm, and each of the first and second interconnections (111, 112), and each of the first, second, and third connections (114, 121, and 131) has a width about 2 mm.

9. The endoscopic device (100) of claim 5, further comprising a fourth unit (14), the fourth unit (14) comprising a bridge point (14a) and a connecting point (14b), both being on a periphery thereof, a third interconnection (113) interconnecting a third bridge point (11c) of the first unit (11) and the bridge point (14a) of the fourth unit (14), a fourth connection (141) comprising a first section (141a) extending from the connecting point (14b) of the fourth unit (14), and a second section (141b) extending from the first section (141a) with a fourth passive electronic component (82) is posed thereon, wherein the fourth unit (14) is disposed under the third unit (13) further away from the second unit by bending the fourth interconnection (113) so as to shape the circuit board (10B) into a four layered structure.

10. The endoscopic device (100) of claim 9, wherein the first sections (121a, 114a) both have a length greater than a distance between the first unit (11) and the second unit (12); the first section (131a) has a length greater than a distance between the first unit (11) and the third unit (13); the first section (141a) has a length greater than a distance between the first unit (11) and the fourth unit (14).

11. The endoscopic device (100) of claim 9, further comprising a second active electronic component (61) disposed on the second unit (12), a third active electronic component (71) disposed on the third unit (13), and a fourth active electronic component (81) disposed on the fourth unit (14).

12. The endoscopic device (100) of claim 9, wherein each of the first, second, and third interconnections (111, 112, and 113), each of the first, second, third, and fourth connections (114, 121, 131, and 141) has a width about 2 mm, and the cover (40) has a diameter less than 5 mm.

13. An endoscopic device (100) comprising:
an image capturing assembly (20) comprising a seat (22) and a lens (21) disposed on the seat (22);
a light source (30) disposed in relationship to the seat (22) for light emission;
a flexible circuit board (10A) comprising:
a first unit (11) having a first side and an opposing second side, the first unit comprising a first active electronic component (51) and the image capturing assembly (20) disposed on the first side for image capture and processing, the first unit (11) further comprising a first bridge point (11a) and a connecting point (11d) spaced around a periphery thereof;
a second unit (12) comprising first and second bridge points (12a, 12c) and a connecting point (12b), both being on a periphery thereof;
a third unit (13) comprising a first bridge point (13a) and a connecting point (13b), both being on a periphery thereof;
a first interconnection (111) interconnecting the first bridge point (11a) of the first unit (11) and the first bridge point (12a) of the second unit (12);
a second interconnection (112) interconnecting the second bridge point (12c) of the second unit (12) and the first bridge point (13a) of the third unit (13);
a first connection (114) comprising a first section (114a) extending from the connecting point (11d) of the first unit (11), and a second section (114b) extending from the first section (114a) with a first passive electronic component (52) disposed thereon;
a second connection (121) comprising a first section (121a) extending from the connecting point (12b) of the second unit (12), and a second section (121b) extending from the first section (121a) with a second passive electronic component (62) disposed thereon;
a third connection (131) comprising a first section (131a) extending from the connecting point (13b) of the third unit (13), and a second section (131b) extending from the first section (131a) with a third passive electronic component (72) disposed thereon;
wherein the second unit (12) is disposed under the first unit (11), facing the second side of the first unit, by bending the first interconnection (111), the third unit (13) is disposed under the second unit (12) further away from the first unit by bending the second interconnection (112) so as to shape the circuit board (10A) as into a three layered structure;
a cover (40) including a transparent member (41), facing the first side of the first unit for covering the three layered structure, and aligned with the image capturing assembly (20).

14. The endoscopic device (100) of claim 13, wherein the first section (121a, 114a) both have a length greater than a distance between the first unit (11) and the second unit (12); the first section (131a) has a length greater than a distance between the first unit (11) and the third unit (13); the first section (141a) has a length greater than a distance between the first unit (11) and the fourth unit (14).

15. The endoscopic device (100) of claim 13, further comprising a second active electronic component (61) disposed on the second unit (12); a third active electronic component (71) disposed on the third unit (13); a fourth active electronic component (81) disposed on the fourth unit (14).

16. The endoscopic device (100) of claim 13, wherein each of the first, second, and third interconnections (111, 112, and 113), each of the first, second, third, and fourth connections (114, 121, 131, and 141) has a width about 2 mm, and the cover (40) has a diameter less than 5 mm.

17. The endoscopic device (100) of claim 13, further comprising a fourth unit (14), the fourth unit (14) comprising a bridge point (14a) and a connecting point (14b), both being on a periphery thereof, a third interconnection (113) interconnecting a second bridge point (13c) of the third unit (13) and the bridge point (14a) of the fourth unit (14), a fourth connection (141) comprising a first section (141a) extending from the connecting point (14b) of the fourth unit (14), and a second section (141b) extending from the first section (141a) with a fourth passive electronic component (82) is posed thereon, wherein the fourth unit (14) is disposed under the third unit (13) further away from the second unit by bending the fourth interconnection (113) so as to shape the circuit board (10B) into a four layered structure.

18. The endoscopic device (100) of claim 17, wherein the first section (121a, 114a) both have a length greater than a distance between the first unit (11) and the second unit (12); the first section (131a) has a length greater than a distance between the first unit (11) and the third unit (13); the first section (141a) has a length greater than a distance between the first unit (11) and the fourth unit (14).

19. The endoscopic device (100) of claim 17, further comprising a second active electronic component (61) disposed on the second unit (12); a third active electronic component (71) disposed on the third unit (13); a fourth active electronic component (81) disposed on the fourth unit (14).

20. The endoscopic device (100) of claim 17, wherein the cover (40) has a diameter less than 5 mm, and each of the first, second, and third interconnections (111, 112, and 113), each of the first, second, third, and fourth connections (114, 121, 131, and 141) has a width about 2 mm.
